(19) United States
(12) Patent Application Publication
(10) Pub. No.: US 2013/0144272 A1
(43) Pub. Date: Jun. 6, 2013

(54) STRICTURE TREATMENT AND DRAINAGE CATHETER

(71) Applicant: Christopher J. Cutie, Lexington, MA (US)

(72) Inventor: Christopher J. Cutie, Lexington, MA (US)

(21) Appl. No.: 13/704,125

(22) PCT Filed: Jun. 13, 2011
(86) PCT No.: PCT/US11/40121

§ 371 Date: Feb. 22, 2013

Related U.S. Application Data
(60) Provisional application No. 61/354,300, filed on Jun. 14, 2010.

(51) Int. Cl. A61M 25/00

(52) U.S. Cl. CPC A61M 25/0017
USPC 604/544

(57) ABSTRACT

A stricture treatment and drainage catheter comprises a drainage lumen supporting a stricture dilator that is changeable between a collapsed position and a plurality of expanded positions. An expansion indicator operatively connects to the stricture dilator and displays an indication if the stricture dilator is in one of the plurality of expanded positions.

### CROSS-REFERENCE TO RELATED APPLICATION

This application claims the benefit of U.S. Provisional Patent Application No. 61/354,300 filed Jun. 14, 2010, the disclosure of which is hereby incorporated by reference in its entirety.

### STATEMENT CONCERNING FEDERALLY SPONSORED RESEARCH OR DEVELOPMENT

Not applicable.

### FIELD OF THE INVENTION

This invention relates to devices and methods for treating urethral strictures.

### BACKGROUND OF THE INVENTION

Urethral strictures are caused by the growth of scar tissue typically following injury related-trauma and/or a bacterial infection. As the name implies, such scar tissue build-up narrows and thereby makes urination difficult and painful for an individual. In extreme cases, a urethral stricture may close the urethra and prevent an individual from urinating. Urinary retention may result in a medical emergency.

There are currently several methods available to treat urethral strictures. In one such method, the patient is first sedated. A set of progressively larger metal rods are then inserted into the urethra to gradually increase its size. Unfortunately, such a procedure can damage both healthy and diseased tissue, and such procedures are also typically painful despite the use of sedatives. Further still, a drainage catheter is often used after the procedure, in some cases for up to several days, to ensure urine flow is not obstructed by inflammation caused by the procedure.

Another method for treating urethral strictures involves surgically repairing the damaged section of the urethra. This includes using small endoscope attached to a blade for cutting away scar tissue. However, such a procedure is technically challenging and may fail due to excessive urethral bleeding.

Urethroplasty is yet another method for treating urethral strictures. This procedure involves making a large incision to remove scar tissue followed by realignment of native tissues or grafting a biocompatible material. Unfortunately, urethroplasty requires significant operating time and inpatient treatment. Patients must also use a urinary catheter for weeks after the procedure and may often experience significant pain and discomfort during that period. Furthermore, these procedures require skilled anesthesia and surgical personnel, and as such, may cost up to several thousand dollars.

Considering the limitations of the previous devices and methods, new devices and methods for treating urethral strictures are needed. Such devices and methods advantageously reduce patient discomfort, permit urine flow during a procedure, reduce urethral inflammation compared to other treatment devices and methods, reduce the need for patient sedation, and/or increase the feasibility of treating urethral strictures outside of hospital settings.

### SUMMARY OF THE INVENTION

In one aspect, the present invention provides a stricture treatment and drainage catheter comprising a drainage lumen having a first end and a second end. The first end defines a drainage inlet and the second end defines a drainage outlet. The catheter further comprises a stricture dilator supported by the drainage lumen between the second end and the first end. The stricture dilator is changeable between a collapsed position and a plurality of expanded positions. An expansion indicator operatively connects to the stricture dilator and displays an indication if the stricture dilator is in one of the plurality of expanded positions.

In some embodiments, the expansion indicator displays the expanded size of the stricture dilator as a French scale number. Furthermore, in some embodiments, the stricture dilator comprises a radially expanding mandrel having a plurality of mandrel legs. Each of the mandrel legs has a second end and a first end, and each of the mandrel legs bows outwardly between the second end and the first end in each of the plurality of expanded positions. In other embodiments, the stricture dilator comprises an inflatable stricture dilator that defines a second internal chamber, and the second internal chamber receives a second substance to expand the inflatable stricture dilator.

The foregoing and advantages of the invention will appear in the detailed description which follows. In the description, reference is made to the accompanying drawings which illustrate a preferred embodiment of the invention.

### BRIEF DESCRIPTION OF THE DRAWINGS

The invention will hereafter be described with reference to the accompanying drawings, wherein like reference numerals denote like elements, and:

FIG. 1 is a side view of a stricture treatment and drainage catheter according to the present invention; a stricture dilator of the catheter is shown in a collapsed position;

FIG. 2 is a longitudinal section view of the catheter of FIG. 1;

FIG. 3 is a side view of the catheter of FIG. 1 with the stricture dilator in an expanded position;

FIG. 4 is a longitudinal section view of the catheter with the stricture dilator supported directly by an anchoring lumen;

FIG. 5 is a side view of a second embodiment of a stricture treatment and drainage catheter according to the present invention; a stricture dilator of the catheter is shown in a collapsed position;

FIG. 6 is a side view of the catheter of FIG. 5 with the stricture dilator in an expanded position;

FIG. 7 is a longitudinal section view of the catheter of FIG. 5;

FIG. 8 is a schematic diagram of the catheter of FIG. 5 including a pump and a processor;

FIG. 9 is a side view of a third embodiment of a stricture treatment and drainage catheter according to the present invention; a stricture dilator of the catheter is shown in a collapsed position; and

FIG. 10 is a side view of the catheter of FIG. 9 with the stricture dilator in an expanded position.

### DETAILED DESCRIPTION OF THE PREFERRED EMBODIMENT

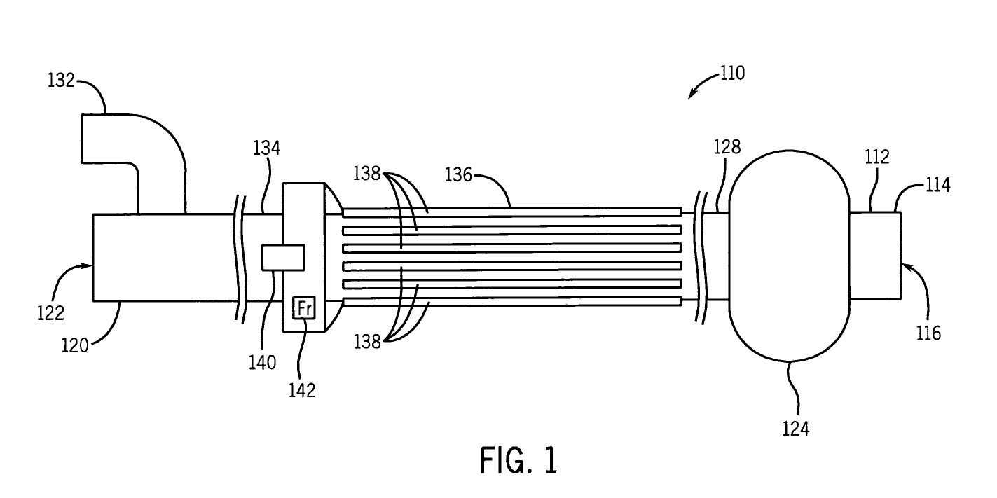

Referring first to FIGS. 1-3, a stricture treatment and drainage catheter 110 according to the present invention permits urinary drainage from a subject's bladder while simultaneously relieving a urethral stricture (i.e., dilating the urethra at a urethral stricture). As described in further detail below, the device 110 includes components that are expandable to various sizes appropriate for treating strictures of varying severity, or to progressively expand the stricture during treatment (e.g., over the course of several days).

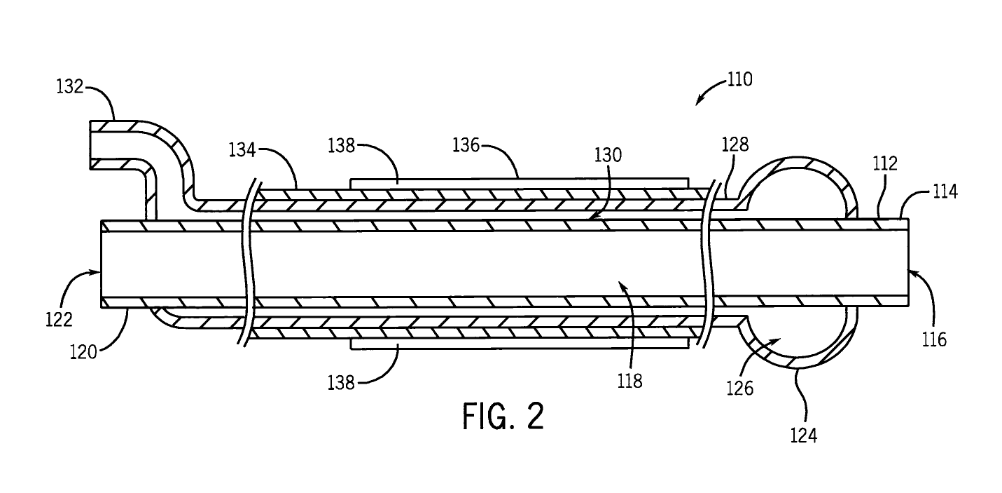

Still referring to FIGS. 1-3, the device 110 includes a first or drainage lumen 112 to provide the aforementioned urinary drainage. That is, the drainage lumen 112 has a first end 114 that defines a drainage inlet 116 for receiving urine from a subject's bladder. Urine is directed through a drainage passageway 118 (FIG. 2) toward a second end 120 of the lumen 112. Urine exits the device through a drainage outlet 122 at the second end 120 of the lumen 112. The drainage outlet 122 may include appropriate couplings or fittings (not shown) to connect the drainage lumen 112 to waste disposal containers or the like.

The drainage lumen 112 may comprise any of a variety of materials commonly associated with drainage catheters, such as flexible polymers. However, other materials or combinations of materials may also be used without departing from the scope of the invention.

In addition to drainage components, the catheter 110 also includes components to anchor or prevent the device from being unintentionally withdrawn from the urethra. Specifically, the catheter 110 includes an inflatable anchor 124 (e.g., an expandable polymer balloon) proximate the first end 114 of the drainage lumen 112. The inflatable anchor 124 defines an internal anchor chamber 126 (FIG. 2) that receives an anchoring substance (e.g., a liquid, gas, or hydrogel) to expand or inflate the anchor 124. In operation, the inflatable anchor 124 is positioned within the subject's bladder and inflated to a size larger than the diameter of the urethra. As such, the anchor 124 cannot pass through the urethra when inflated and thereby prevents the device 110 from being unintentionally withdrawn.

The inflatable anchor 124 is supported by a second or anchoring lumen 128 disposed about the drainage lumen 112. The anchoring lumen 128 is spaced apart from the drainage lumen 112 and thereby defines an anchoring passageway 130 (FIG. 2) through which the anchoring substance may pass and enter the internal anchor chamber 126 of the inflatable anchor 124. The anchoring substance may also return from the chamber 126 through the anchoring passageway 130 to an anchor port 132 to collapse or deflate the anchor 124. The anchor port 132 may receive and deliver the anchoring substance to a port or valve of a delivery apparatus (not shown) configured to supply the anchoring substance to the device 110 (e.g., a pump). As such, the anchor port 132 may include appropriate couplings or fittings (not shown) to connect the anchoring lumen 128 to the delivery apparatus.

The inflatable anchor 124, the anchoring lumen 128, and the anchor port 132 may comprise any of a variety of materials commonly associated with drainage catheters, such as flexible polymers. However, other materials or combinations of materials may also be used without departing from the scope of the invention.

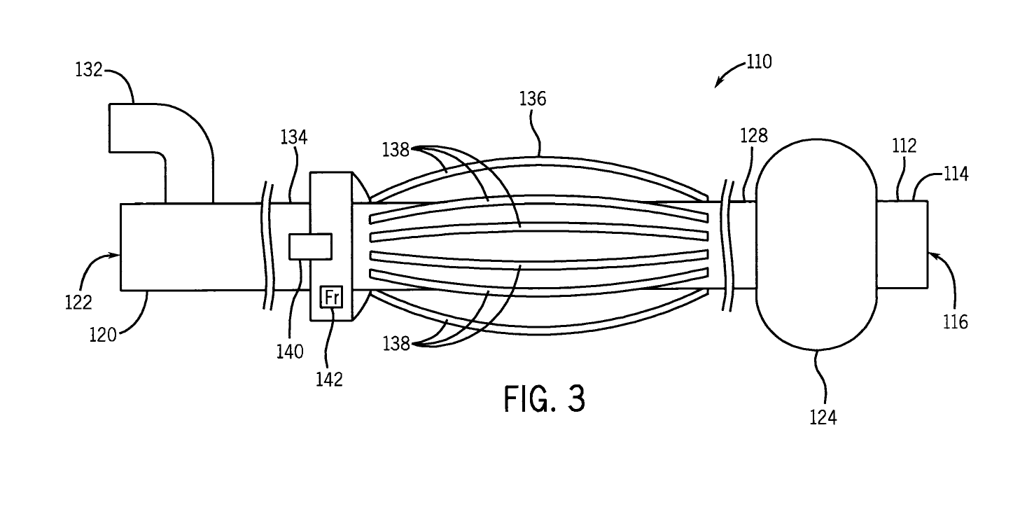

As the name implies, the stricture treatment and drainage catheter 110 also includes components that engage and increase the size of the urethra at a urethral stricture. Specifically, the device 110 includes a third or dilator lumen 134 disposed about the anchoring lumen 128. The dilator lumen 134 supports a stricture dilator that is expandable to engage and relieve a urethral stricture. In the embodiment shown in FIGS. 1-3, the stricture dilator is a radially expanding mandrel 136 disposed between the first and second ends of the device. The expanding mandrel 136 comprises a plurality of mandrel legs 138 that are movable from a collapsed position (an unloaded position, FIG. 1) to a plurality of expanded positions (a loaded position, e.g., FIG. 3) and vice versa.

In the expanded positions, the mandrel legs 138 define a larger overall size (i.e., the outer perimeter defined by the outermost points of the mandrel legs 138) than in the collapsed position. Stated another way, each of the mandrel legs 138 has a first end that is movable along the dilator lumen 134 and a second end that is fixed to the dilator lumen 134. In the expanded positions, the mandrel legs 138 bow outwardly between the first end and the second end to define a larger overall size than when the device 110 is in the collapsed position.

The mandrel legs 138 may comprise any of a variety of flexible and fatigue-resistant materials, such as silicone, latex, polyethylene, polytetrafluoroethylene Teflon®), or the like. However, other materials or combinations of materials may also be used without departing from the scope of the invention.

The mandrel legs 138 operatively connect to an expansion control positioned toward the second end of the device 110. In the embodiment shown in FIGS. 1-3, the expansion control is a rotatable knob or dial 140, and rotation of the dial 140 moves the mandrel legs 138 from the collapsed position to the expanded positions and vice versa. The mandrel legs 138 and the dial 140 are also operatively connected to an expansion indicator 142 that displays the overall expanded size of the mandrel legs 138.

In its simplest form, the expansion indicator 142 is a rotatable wheel that displays the French scale number of the overall expanded size of the mandrel legs 138. In other embodiments, the expansion indicator 142 may be a digital display. Regardless of the specific form of the expansion indicator 142, the dial 140 and the mandrel legs 138 may be connected such that a single turn or an increment of a turn of the dial 140 provides a discrete size change of the overall size of the mandrel legs 138. For example, one turn or one-half turn of the dial 140 may cause the overall size of the mandrels legs 138 to change by one French scale number. In any case, the expansion indicator 142 permits medical personnel to precisely control the overall size of the mandrel legs 138. As such, the overall size of the mandrel legs 138 can be adjusted for urethral strictures of varying severity or in view of other factors, such as subject age and overall wellness. These considerations can reduce subject discomfort and increase the effectiveness of treatment using the device 110.

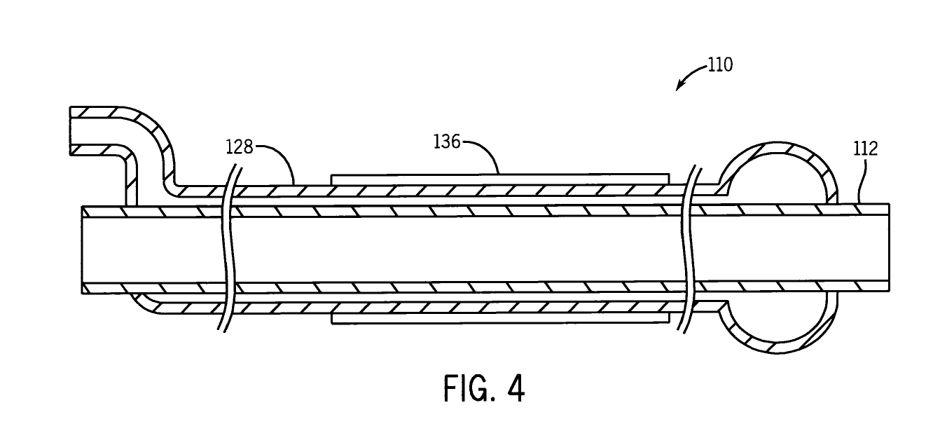

Of course, the device 110 may vary from the above description without departing from the scope of the invention. For example and as described above, the stricture dilator is supported by a dilator lumen 134. In some embodiments, the dilator lumen 134 may be detachably supported by the anchoring lumen 128. As such, the dilator lumen 134 and the stricture dilator may be detached from the anchoring lumen 128 after use. The dilator lumen 134 and the stricture dilator could then be sterilized for subsequent use with another anchoring lumen 128 and drainage lumen 112. As another example and referring to FIG. 4, the stricture dilator may connect directly to the outside of the anchoring lumen 128 instead of connecting to a dilator lumen 134. As yet another example, the dial 140 may be remotely located relative to the expanding mandrel 136 (e.g., proximate the anchor port 132). As yet another example, rotation of the dial 140 may only move the mandrel legs 138 from the collapsed position towards the expanded positions (i.e., only increase the overall size of the mandrel legs 138). In this case, the device 110 may include a separate release mechanism (not shown) that is actuatable to permit the mandrel legs 138 to return to the collapsed position by simply moving toward their unloaded position.

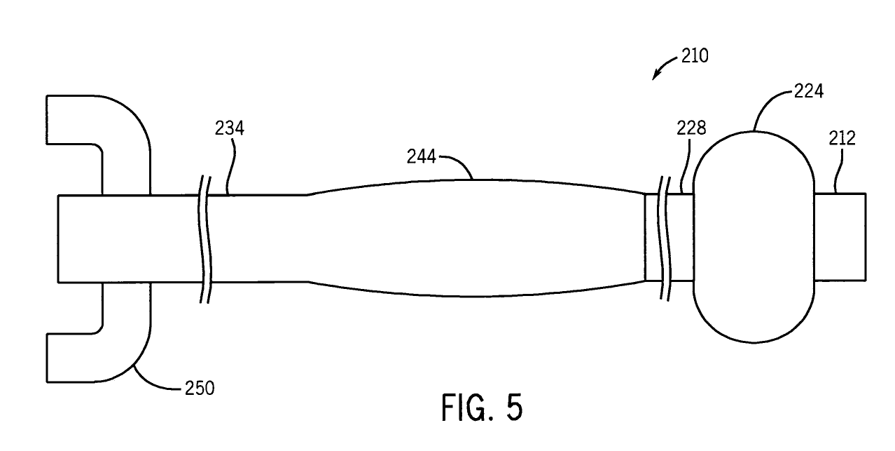

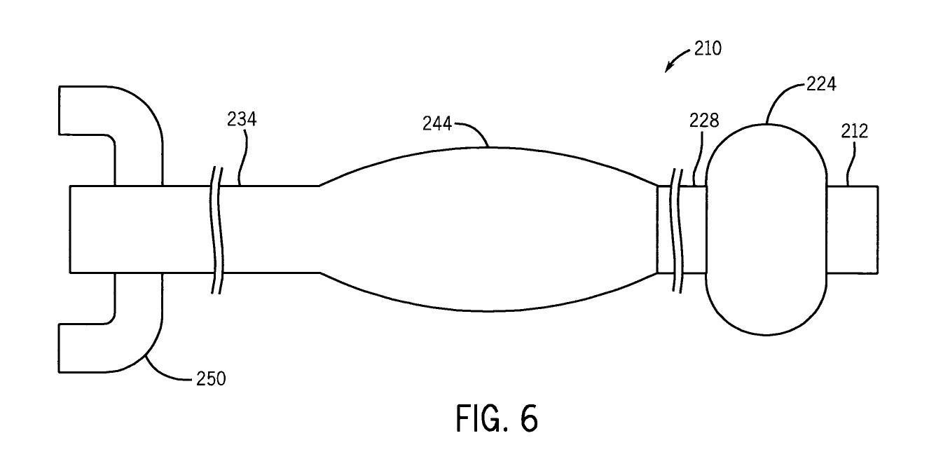

Referring now to FIGS. 5-8, a second embodiment of a stricture treatment and drainage catheter 210 of the present invention includes a drainage lumen 212, an anchoring lumen 228, and an inflatable anchor 224 as described above. The dilator lumen 234 also supports a stricture dilator between the first and second ends of the device 210. Unlike the device described above, however, the stricture dilator is an inflatable stricture dilator 244 that is movable from a collapsed position (FIG. 5) to a plurality of expanded positions (e.g., FIG. 6) and vice versa. In the expanded positions, the inflatable stricture dilator 244 engages and relieves a urethral stricture.

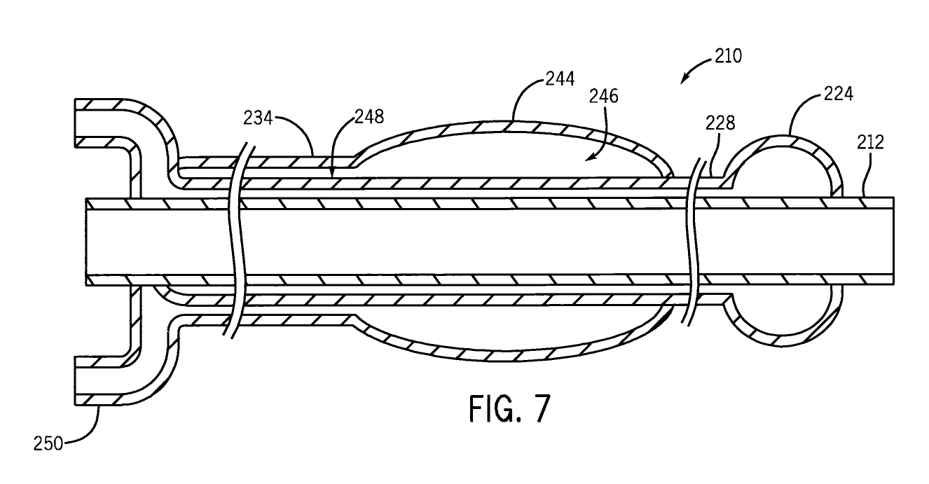

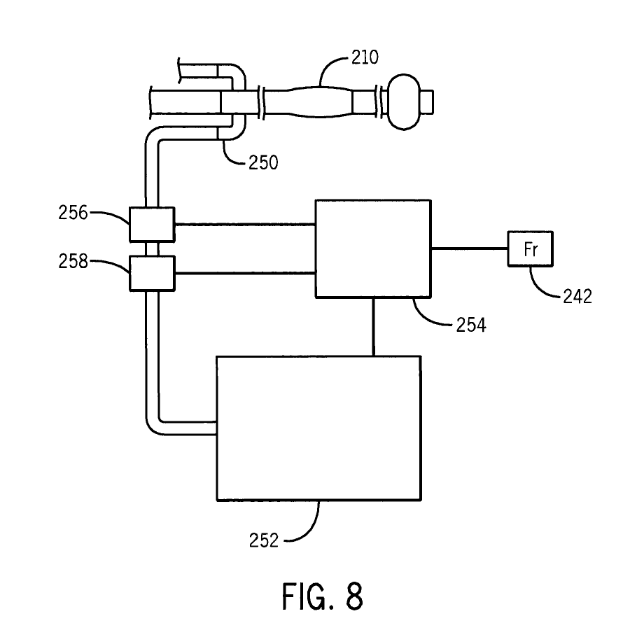

As shown most clearly in FIG. 7, the inflatable stricture dilator 244 defines an internal dilator chamber 246 that receives a dilator substance (e.g., a liquid, gas, or hydrogel) to expand or inflate the stricture dilator 244. The dilator lumen 234 is also spaced apart from the drainage lumen 212 and thereby defines a passageway 248 through which the dilator substance may pass and enter the chamber 246. The dilator substance may also return from the chamber 246 through the passageway 248 to a dilator port 250 to collapse or deflate the stricture dilator 244. The dilator port 250 may receive and deliver the dilator substance to a port or valve of a delivery apparatus 252 (e.g., a pump, FIG. 8) configured to supply the dilator substance to the device 210. As such, the dilator port 250 may include appropriate couplings or fittings to connect the dilator lumen 234 to the delivery apparatus 252.

As shown schematically in FIG. 8, the delivery apparatus 252 may be controlled by a processor 254. The processor 254 operatively connects to a flow rate sensor 256 and a pressure sensor 258 coupled between the delivery apparatus 252 and the catheter 210. As such, the processor 254 is capable of determining the amount of the dilator substance provided to the device 210 and the pressure of the dilator substance within the device 210. The processor 254 may use this information, as well as initial dimensions of the stricture dilator 244, to determine the overall expanded size of the stricture dilator 244. The processor 254 may also operatively connect to the expansion indicator 242 to display the overall expanded size of the stricture dilator 244.

In some cases, the delivery apparatus 252 may be the same apparatus that delivers the anchoring substance to the inflatable anchor 224. In these cases, the system shown in FIG. 8 would include additional conduits and/or control valves such that the delivery apparatus 252 could selectively deliver the substance to the inflatable anchor 224 and the stricture dilator 244.

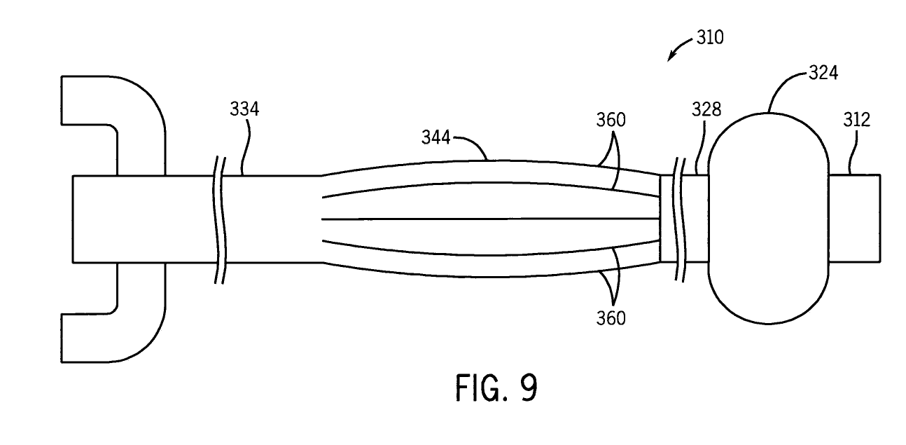

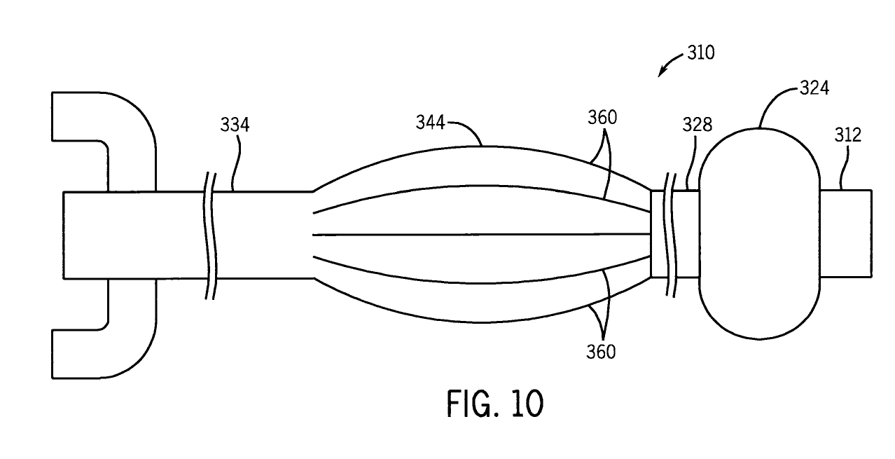

Referring now to FIGS. 9-10, a third embodiment of a stricture treatment and drainage catheter 310 of the present invention includes a drainage lumen 312, an anchoring lumen 328, and an inflatable anchor 324 as described above. The dilator lumen 334 also supports an inflatable stricture dilator 344 between the first and second ends of the device 310. Unlike the device described above, however, the inflatable stricture dilator 344 is defined by a plurality of separate veins 360. Each of the veins 360 is movable between a collapsed position (FIG. 9) and a plurality of expanded positions (e.g., FIG. 10) and vice versa. In the expanded positions, the veins 344 engage and relieve a urethral stricture. The catheter device 310 may be used with liquids, gases, or hydrogels as well as a delivery apparatus, controller, flow rate sensor, and a pressure sensor as described above.

Regardless of the specific construction of the stricture treatment and drainage catheter, the device 110 is preferably used as follows. The second and third embodiments of the device 210 and 310 are also preferably used as follows, although only reference numbers of the first embodiment of the device 110 are used for simplicity. First, if necessary, the dilator lumen 134 is connected to the anchoring lumen 128. Next, the device 110 is inserted into the subject's urethra such that the drainage inlet 116 and the inflatable anchor 124 are disposed within the bladder and the stricture dilator is disposed adjacent the urethral stricture. The device 110 is then connected to a delivery apparatus to provide the anchoring substance to the inflatable anchor 124 and thereby expand the anchor 124. Next, the stricture dilator is moved to one of the expanded positions by rotating the dial 140 or providing the dilating substance to the device. This expansion causes the stricture dilator to contact and relieve a urethral stricture.

The above method may further include additional and optional steps. For example, the overall size of the stricture dilator may be gradually increased over a predetermined time period (e.g., several days). In some embodiments, this may be controlled automatically by the processor 254. As another example, the stricture dilator may be expanded to deploy a stent that engages and treats a urethral stricture. The device may then be removed while the stent remains in place in the urethra.

From the above description, it should be apparent that all of the embodiments of the stricture treatment and drainage catheter provide several common advantages. For example, the device permits urine flow during a procedure due to the presence of the drainage lumen. As another example, the device reduces subject discomfort and urethral inflammation compared to other treatment devices because the stricture dilator expands radially to engage and treat a stricture. That is, in contrast to the previous devices described above, the stricture treatment and drainage catheter does not traumatically engage both diseased and healthy urethral tissue during insertion. This aspect of the device also reduces the need for subject sedation. As yet another example, the expansion indicator permits medical personnel to precisely control the expanded size of the stricture dilator, thereby increasing the effectiveness of treatment using the device. Furthermore, in some ways the device is used in a similar manner compared to a drainage catheter, and the expansion indicator facilitates ease of use. As such, methods for using the device are relatively simple compared to the previous stricture treatment methods described above, and therefore the device facilitates treatment of urethral strictures outside of hospital settings.

Furthermore, the embodiments of the stricture treatment and drainage catheter also provide different advantages compared to one another. For example, the device 110 does not require delivery apparatus use (i.e., pump use) after the inflatable anchor 124 is initially expanded. Therefore the pump can be disconnected from the catheter device 110 after the inflatable anchor 124 is initially expanded. A valve or the like would be needed to seal the anchoring substance within the anchoring lumen 128 and the inflatable anchor 124, but nevertheless the pump could be disconnected and put to other uses. Furthermore, the mandrel legs 138 provide relatively large forces for relieving urethral strictures compared to their small size. Other means for providing these forces are typically much larger. As another example, the devices 210 and 310 could be supplied a liquid, gas, or hydrogel from the same pump that delivers the substance to the inflatable anchor. As such, various aspects of treatment could be controlled from the user interface of the pump after the device is inserted into the subject's urethra.

A preferred embodiment of the invention has been described in considerable detail. Many modifications and variations to the preferred embodiment described will be apparent to a person of ordinary skill in the art. Therefore, the invention should not be limited to the embodiment described, but should be defined by the claims that follow.

We claim:

1. A stricture treatment and drainage catheter comprising:
a drainage lumen having a first end and a second end, the first end defining a drainage inlet and the second end defining a drainage outlet;
a stricture dilator supported by the drainage lumen between the second end and the first end, the stricture dilator being changeable between a collapsed position and a plurality of expanded positions; and
an expansion indicator operatively connected to the stricture dilator and displaying an indication if the stricture dilator is in one of the plurality of expanded positions.

2. The stricture treatment and drainage catheter of claim 1, wherein the stricture dilator is removably supported by the drainage lumen.

3. The stricture treatment and drainage catheter of claim 1, wherein the indication displayed by the expansion indicator includes a size of the stricture dilator in the one of the plurality of expanded positions.

4. The stricture treatment and drainage catheter of claim 1, wherein the stricture dilator comprises a radially expanding mandrel having a plurality of mandrel legs, each of the mandrel legs having a second end and a first end, and each of the mandrel legs bowing outwardly between the second end and the first end in each of the plurality of expanded positions.

5. The stricture treatment and drainage catheter of claim 4, further comprising an expansion control in driving engagement with the plurality of mandrel legs and being operable to move the plurality of mandrel legs from the collapsed position to the plurality of expanded positions and vice versa.

6. A method for treating a urethral stricture in a subject, the method comprising:
providing the stricture treatment and drainage catheter of claim 1;
inserting the stricture treatment and drainage catheter into the subject's urethra such that the stricture dilator is disposed adjacent the urethral stricture;
moving the stricture dilator to one of the plurality of expanded positions such that the stricture dilator contacts the urethral stricture; and
gradually increasing a size of the stricture dilator.

7. A stricture treatment and drainage catheter comprising:
a first lumen having a drainage outlet at a second end in fluid communication with a drainage inlet at a first end via a first passageway;
an inflatable anchor supported by the first lumen proximate the first end, the inflatable anchor defining a first internal chamber;
a second lumen disposed about the first lumen, the second lumen defining an second passageway in fluid communication with the first internal chamber;
an inflatable anchor port supported by the first lumen proximate the second end, the inflatable anchor port being in fluid communication with the first internal chamber via the second passageway to deliver a first substance to the first internal chamber to expand the inflatable anchor and receive the first substance from the first internal chamber to collapse the inflatable anchor;
a stricture dilator supported by the first lumen between the second end and the first end, the stricture dilator being changeable between a collapsed position and a plurality of radially expanded positions; and
an expansion indicator operatively connected to the stricture dilator and displaying an expanded size of the stricture dilator when the stricture dilator is in at least some of the plurality of radially expanded positions.

8. The stricture treatment and drainage catheter of claim 7, further comprising a third lumen disposed about the second lumen, the stricture dilator being directly connected to the third lumen.

9. The stricture treatment and drainage catheter of claim 8, wherein the stricture dilator comprises an inflatable stricture dilator that defines a second internal chamber, and the third lumen defines a third passageway in fluid communication with the second internal chamber to deliver a second substance to the second internal chamber to expand the inflatable stricture dilator and receive the second substance from the second internal chamber to collapse the inflatable stricture dilator.

10. The stricture treatment and drainage catheter of claim 9, further comprising a processor operatively connected to the stricture dilator to determine the expanded size of the stricture dilator when the stricture dilator is in at least some of the plurality of radially expanded positions, and the processor being operatively connected to the expansion indicator to display the expanded size on the expansion indicator.

11. The stricture treatment and drainage catheter of claim 10, further comprising:
a flow rate sensor configured to sense a flow of the second substance delivered to the second internal chamber and sending a flow signal corresponding to the flow to the processor;
a pressure sensor configured to sense a pressure of the second substance within the second internal chamber and sending a pressure signal corresponding to the pressure to the processor;
wherein the processor determines the expanded size of the stricture dilator based at least in part on the flow signal and the pressure signal.

12. A stricture treatment and drainage catheter comprising:
a drainage lumen having a drainage outlet at a second end and a drainage inlet at a first end;
an anchoring balloon supported by the drainage lumen proximate the first end, the anchoring balloon defining an internal anchor chamber;
an anchoring lumen disposed about the drainage lumen, the anchoring lumen defining an anchoring substance passageway in fluid communication with the internal anchor chamber;
a balloon port supported by the drainage lumen proximate the second end, the balloon port being in fluid communication with the internal anchor chamber via the anchoring substance passageway to deliver an anchoring substance to the internal anchor chamber to expand the anchoring balloon and receive the anchoring substance from the internal anchor chamber to collapse the anchoring balloon;
a stricture dilator supported by the drainage lumen between the second end and the first end, the stricture dilator being changeable between a collapsed position and a plurality of expanded positions; and
an expansion indicator operatively connected to the stricture dilator and displaying an expanded size of the stricture dilator when the stricture dilator is in at least some of the plurality of expanded positions.

13. The stricture treatment and drainage catheter of claim 12, wherein the expanded size displayed by the expansion indicator is a French scale number.

14. The stricture treatment and drainage catheter of claim 13, further comprising a dilator lumen disposed about the anchoring lumen and defining a third passageway, the stricture dilator being directly connected to the dilator lumen, wherein the stricture dilator comprises an inflatable stricture dilator that defines a second internal chamber, and the third passageway is in fluid communication with the second internal chamber to deliver a second substance to the second internal chamber to expand the inflatable stricture dilator and receive the second substance from the second internal chamber to collapse the inflatable stricture dilator.

15. The stricture treatment and drainage catheter of claim 14, wherein the inflatable stricture dilator is defined by a plurality of separate veins.
